(19) United States
(12) Patent Application Publication
(10) Pub. No.: US 2025/0366794 A1
(43) Pub. Date: Dec. 4, 2025

(54) HEAD ACCELERATION EVENT CLASSIFICATION

(71) Applicant: Prevent Biometrics, Inc., Edina, MN (US)

(72) Inventors: Adam Bartsch, Chaska, MN (US); Jason C. Thibado, Seattle, WA (US)

(21) Appl. No.: 19/222,290

(22) Filed: May 29, 2025

Related U.S. Application Data
(60) Provisional application No. 63/654,386, filed on May 31, 2024.

(51) Int. Cl. A61B 5/00; A61B 5/11

(52) U.S. Cl. CPC A61B 5/7282; A61B 5/1114; A61B 5/1121; A61B 5/682; A61B 5/7203; A61B 5/725; A61B 5/7267; A61B 2503/10; A61B 2562/0219

(57) ABSTRACT

A monitoring device system can include a signal receiver circuit which can be configured to receive kinematic information of a user, the kinematic information including impact data corresponding to a head acceleration event (HAE). The system can also include an assessment circuit which can be configured to process the impact data, including to classify the HAE based on one or more features indicative of a noise level of the HAE, and based upon the classification, pass the impact data through a corresponding filter to generate filtered impact data.

### CROSS-REFERENCE TO RELATED PATENT DOCUMENTS

This patent application claims the benefit of priority to U.S. Application Ser. No. 63/654,386, filed May 31, 2024, which application is also related to Bartsch et al., U.S. patent application Ser. No. 17/731,553 entitled “PROXIMITY SENSOR TECHNIQUES,” filed on Apr. 28, 2022 (Attorney Docket No. 5249.025US1) both of which are hereby incorporated by reference herein in their entirety.

### TECHNICAL FIELD

The present disclosure relates to systems for impact monitoring. More particularly, the present disclosure relates to systems for accurately sensing impacts to the human head or body and accounting for and/or screening out false positive results.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Sensing head impacts for purposes of assessing risk of brain damage and/or trauma (e.g., concussions) has come to the forefront in many activities. Sensor systems on helmets, on skin patches, on mouth guards, or on other systems or devices have been studied and implemented. Several difficulties can exist with respect to obtaining accurate and precise results. For example, helmets are designed to reduce and/or distribute impact loads to the head via a relatively loose helmet-to-head coupling, so sensors on the helmet may sense impacts that are higher or otherwise different than those that are passed onto the head and the direction and/or magnitude of the impact on the helmet may create uncertainty as to the forces experienced by the head. One particular difficulty with respect to obtaining accurate and precise results across many systems relates to false positives. For example, impacts may be sensed by equipment when a user drops the equipment or drops or sets down a bag that the equipment is in. Still other impacts may be sensed when a bag is being carried and swings against an obstruction. These and other impacts that may be sensed by an impact sensor are not relevant to head impacts and, preferably, would be screened out of the data that is collected, as opposed to being reported as head acceleration events (HAEs).

Some preliminary efforts to rule out false positives for mouthguard-based systems have focused on assuring that the mouth guard is positioned in an engaged position on the teeth of a user (e.g., a wearer). For example, a proximity sensor has been suggested as a method for determining when the mouthguard is on the teeth. However, when not in use, users have been known to turn the mouthguard sideways and chew on it, which may trigger the proximity sensor(s) and result in false positive readings. Also, a user may put a finger, lip, or article of clothing in front of the sensor causing the system to believe, so to speak, that it is on the teeth. Simple on/off switches have also been suggested, but may only be helpful to the extent the device is turned off when not being actively used during situations where impact results are desired. (i.e., during a game when the player is not on the field and has his/her helmet off or mouthguard out of the mouth). Counting on users to constantly activate and deactivate sensors might not be reliable.

Further, data related to an HAE can be affected by noise or can include data that does not accurately represent the impact experienced by the head of the user. For example, if a user is struck in the teeth while wearing an impact sensing wearable mouthguard, the acceleration experienced by the mouthguard may not be representative of the acceleration experienced by the center of gravity of the user's head.

### SUMMARY

In an example, a monitoring device system can include a signal receiver circuit which can be configured to receive kinematic information of a user, the kinematic information including impact data corresponding to a head acceleration event (HAE). The system can also include an assessment circuit which can be configured to process the impact data, including to classify the HAE based on one or more features indicative of a noise level of the HAE, and based upon the classification, pass the impact data through a corresponding filter to generate filtered impact data.

In an example, a method for classifying a head acceleration event (HAE) can include receiving kinematic information of a user, the kinematic information including impact data corresponding to the HAE. The method can also include classifying the HAE based on one or more features indicative of a noise level of the HAE. The method can also include filtering the impact data through a filter corresponding to the classification to generate filtered impact data.

In an example, a monitoring device system including a wearable mouthguard can include a signal receiver circuit which can be configured to receive proximity data from a proximity sensor in the wearable mouthguard. The monitoring device system can also include an assessment circuit which can be configured to process proximity data from the wearable mouthguard and determine whether the wearable mouthguard can be engaged on the teeth of a user, including to determine a “on-tooth” value of the proximity sensor, and compare the proximity data to the “on-tooth” value.

### BRIEF DESCRIPTION OF THE DRAWINGS

In the drawings, which may not be drawn to scale, like numerals may describe substantially similar components throughout one or more of the views. Like numerals having different letter suffixes may represent different instances of substantially similar components. The drawings illustrate generally, by way of example but not by way of limitation.

FIG. 1 is a perspective view of athletes wearing an impact sensor, according to one or more embodiments, and experiencing bodily impacts while participating in an athletic event.

FIG. 2 is a diagram of a motion sensing device in communication with an outside computing device via one or more communication systems, according to one or more embodiments.

FIG. 3 shows a diagram depicting an example of a method for determining whether an impact sensing device is in an engaged state.

FIG. 4A shows an example of field collected proximity sensor values over time.

FIG. 4B shows an example of a histogram of the field collected proximity sensor values of FIG. 4A.

FIG. 5A shows an example of the proximity sensor values of FIG. 4A near an impact event.

FIG. 5B shows an example of the proximity sensor values of FIG. 4A near an impact event.

FIG. 6 shows a diagram depicting an example of a method for classifying impact data.

FIG. 7A shows an example of unfiltered linear acceleration values.

FIG. 7B shows an example of unfiltered angular velocity values.

FIG. 8A shows an example of head center of gravity linear acceleration data generated from the data of FIG. 7A and FIG. 7B and filtered using a 50 Hz corner frequency filter.

FIG. 8B shows an example of head center of gravity linear velocity data generated from the data of FIG. 7A and FIG. 7B and filtered using a 50 Hz corner frequency filter.

FIG. 8C shows an example of head center of gravity angular acceleration data generated from the data of FIG. 7A and FIG. 7B and filtered using a 50 Hz corner frequency filter.

FIG. 8D shows an example of head center of gravity angular velocity data generated from the data of FIG. 7A and FIG. 7B and filtered using a 50 Hz corner frequency filter.

FIG. 9A shows an example of head center of gravity linear acceleration data generated from the data of FIG. 7A and FIG. 7B and filtered using a 100 Hz corner frequency filter.

FIG. 9B shows an example of head center of gravity linear velocity data generated from the data of FIG. 7A and FIG. 7B and filtered using a 100 Hz corner frequency filter.

FIG. 9C shows an example of head center of gravity angular acceleration data generated from the data of FIG. 7A and FIG. 7B and filtered using a 100 Hz corner frequency filter.

FIG. 9D shows an example of head center of gravity angular velocity data generated from the data of FIG. 7A and FIG. 7B and filtered using a 100 Hz corner frequency filter.

FIG. 10A shows an example of head center of gravity linear acceleration data generated from the data of FIG. 7A and FIG. 7B and filtered using a 200 Hz corner frequency filter.

FIG. 10B shows an example of head center of gravity linear velocity data generated from the data of FIG. 7A and FIG. 7B and filtered using a 200 Hz corner frequency filter.

FIG. 10C shows an example of head center of gravity angular acceleration data generated from the data of FIG. 7A and FIG. 7B and filtered using a 200 Hz corner frequency filter.

FIG. 10D shows an example of head center of gravity angular velocity data generated from the data of FIG. 7A and FIG. 7B and filtered using a 200 Hz corner frequency filter.

FIG. 11A shows an example of filtering peak linear acceleration.

FIG. 11B shows an example of filtering peak linear velocity.

FIG. 12 is a block diagram of an example of portions of a machine upon which one or more portions of the present disclosure may be implemented.

### DETAILED DESCRIPTION

The present application, in one or more embodiments, relates to impact sensing and, in particular, to methods for ruling out false positive readings on impact sensors and for determining a value associated with a HAE. The sensors may be used for sensing impacts to athletes, military personnel, and/or other users. The ability to rule out false positives may allow for a better ability to focus on meaningful impact data and develop and/or generate meaningful protocols or other systems for identifying injury-inducing impacts or a series of impacts that collectively can cause injury. Moreover, the ability to identify relevant impact data amidst an array of otherwise comingled data may allow for further uses beyond injuries that are caused by impacts. Accurately distinguishing between false positives and true HAEs can help to avoid unnecessary interruption of games, sporting events, or other activities (e.g., due to reporting a concussion assessment level impact that was in fact a false positive not related to a HAE), which can help to develop trust of the various users of the impact sensing system. Accurately analyzing true HAEs can be desirable because it can provide a value with a specified level of accuracy.

In an approach, a machine learning model can be used to determine whether or not a signal received from a sensor represents a true HAE (e.g., whether the signal represents a head impact or another event, such as the sensor being dropped on the ground), or rather, a false positive. However, this can be unreliable, which can lead to some non-HAEs (e.g., false positives) being determined to be true HAEs. For example, in some cases, the signal generated by a non-HAE can indistinguishably or nearly indistinguishably match the signal generated by a true HAE.

A system or method can be used to determine whether the wearable device (e.g., wearable mouthguard) is being worn by the user. The machine learning model can be used following the determination that the wearable device is being worn (e.g., and data generated by the device can be related to a true HAE). For example, a sensor (e.g., a proximity sensor) can provide data that can be analyzed to determine whether a wearable device is being properly worn, and therefore, generating data related to true HAEs. After an impact is determined to be a true HAE, a machine learning model can be used to classify data from an impact, which can include classifying the data based on a noise level (e.g., signal-to-noise ratio) of the data. Based on the classification, the impact data can pass through a corresponding filter, and the filtered impact data can be used to determine a value of the impact. This filtering of the impact data based on a noise level of the data can provide a more accurate value related to the impact.

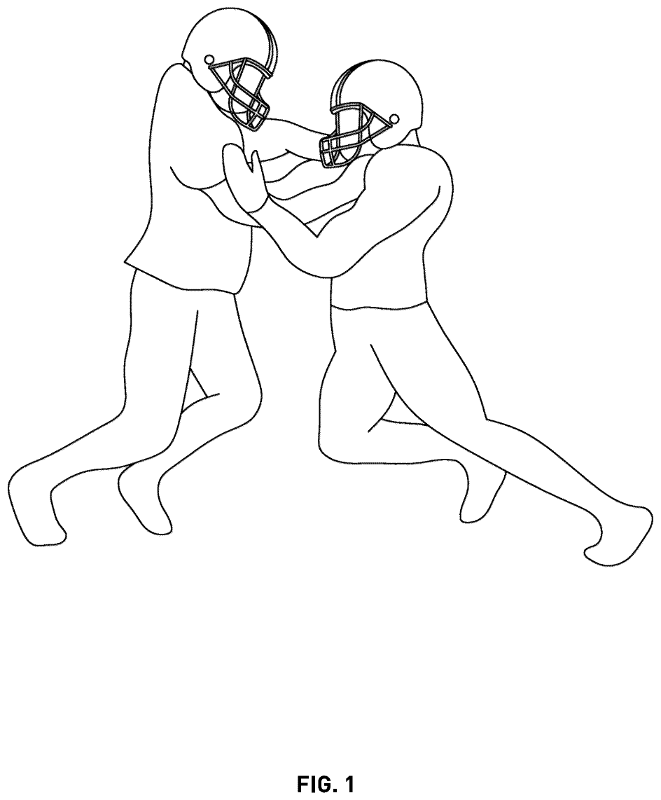

FIG. 1 is a perspective view of athletes wearing an impact sensor, according to one or more embodiments, and experiencing bodily impacts while participating in an athletic event. One or both of the athletes shown can be wearing an impact sensor (e.g., a wearable mouthguard). As shown in FIG. 1, two linemen are engaged with one another in a pushing match commonly occurring throughout a football game. The offensive lineman may be protecting the quarterback, for example, and/or attempting to create a hole for a running back to run through. The defensive lineman may be attempting to get passed the offensive lineman to tackle or, otherwise, interfere with the quarterback or the defensive lineman may be attempting to reach and tackle a running back if the ball has been handed off. Throughout this pushing and shoving exchange, multiple impacts may be sensed by an impact sensor on, for example, a mouthguard of one or both linemen. Moreover, higher speed impacts may occur when a ball carrier is impacted for a tackle or during special teams play where players are running more directly at one another prior to impact.

When the athletes are not engaged in game play, such as between plays or when they are out of the game, their impact sensors may still be active. However, data generated by the impact sensor may not correlate to true HAEs during gameplay. As discussed in more detail below, one or more of separating true HAEs from non-HAEs (e.g., by determining when the wearable sensor is being properly worn), classifying HAEs based on their noise level (e.g., using a machine learning model), or filtering the data (e.g., with a filter corresponding to the determined class) can be helpful in making one or more determinations about the athletes, such as including whether or not the athletes need to be removed from gameplay.

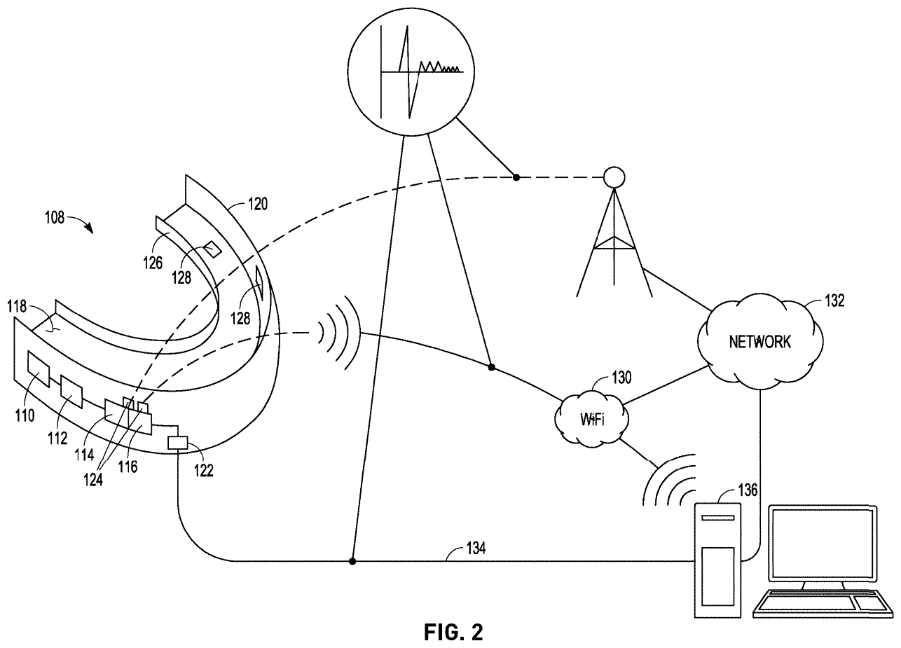

Referring now to FIG. 2, a motion sensing device 108 worn by one or both linemen is shown. The sensing device may be adapted to sense impacts, store and/or analyze the impacts, and transmit the impact data to one or more additional devices. In one or more embodiments, the sensing device may be in the form of a mouthguard configured for kinematic sensing such as a mouthguard worn by athletes during athletic events and having sensing equipment thereon for sensing head impacts. In one or more embodiments, the sensing device 108 may include a body portion and an electronic system including a power source 110, one or more sensors 112, a data storage medium 114, a processor 116, input/output devices 122, and/or receiving and transmitting systems 124.

The body portion may be in the form of a mouthguard or an alternative wearable device may be used. The alternative wearable device may be, for example, a body or skin patch, a clothing patch, a head band, mouthpiece, earpiece, or other wearable device. In the case of a mouthguard, the mouthguard may include a dentition portion 118, a labial portion 120, and a lingual portion 126. The dentition portion 118 may be generally flat and u-shaped and adapted for resting on and/or being positioned between the crown of the teeth. In one or more embodiments, the dentition portion 118 may be adapted for molding to the teeth using a heating and biting process or the dentition portion may be custom fitted and molded, for example. The dentition portion may include an inner u-shaped edge and an outer u-shaped edge. The labial portion 120 may extend upwardly and/or downwardly from the outer u-shaped edge of the dentition portion and may be configured to protect the labial surface of the upper and/or lower teeth. The lingual portion 126 may be provided extending upward and/or downward from the inner u-shaped edge of the dentition portion and may be configured to keep the tongue from slipping between the teeth, for example.

The electronic system may be arranged on the surface of, lodged within, molded within, or otherwise associated with the body portion. In one or more embodiments, the electronics system may be over-molded within the labial or lingual portion of the mouthguard. In one or more embodiments, the mouthguard may be manufactured consistent with the system and methods described in U.S. patent application Ser. No. 16/682,656, filed on Nov. 13, 2019, and entitled Impact Sensing Mouthguard, the content of which is hereby incorporated by reference herein in its entirety. Still other approaches to manufacturing the mouthguard may be used.

The power source 110 may be an electric power source configured for providing power to the sensors, the storage medium, and the processor. In one or more embodiments, the power source may be in the form of a battery such as a nickel cadmium alloy battery, a metal hydride battery, a lithium ion battery, a microscopic battery, or another battery suitable for powering micro electro-mechanical devices.

The sensors 112 may include sensors adapted for sensing kinematic body motion of an athlete, military personnel, or other human user such as those resulting from bodily impact or collision, for example. In one or more embodiments, the sensors may include accelerometers including linear accelerometers, angular accelerometers, gyroscopes, or other motion sensing micro electro-mechanical devices. In one particular embodiment, a 3-axis linear accelerometer may be provided together with a 3-axis gyroscope. The linear accelerometer may be configured for sensing linear accelerations along x, y, and z axes and the gyroscope may be configured for sensing angular velocities along the same set of x, y, and z axes. In one or more embodiments, manufacturing techniques may be used to align the local axes of the two separate sensors. In other embodiments, mathematical techniques may be used to determine if any axes are out of alignment and to normalize the two sets of data to reflect the same set of axes. In one or more embodiments, the normalization may be performed to correspond with a human anatomy axis, where X may be directed anteriorly, Y may be directed laterally, and Z may be directed upward.

As mentioned, the sensor 112 may include kinematic sensors. That is, the sensors 112 may be adapted for sensing kinematic motion of the human body. As such, the sensors 112 may be designed, sized, and calibrated for sensing a range of accelerations commonly reflected by the motion of an athlete during athletic events and, in particular, during an impact event. For example, the sensors may be calibrated for sensing accelerations having magnitudes ranging from approximately 0 g's (e.g., where 1 g is equal to the gravitational force on earth) to approximately 300 g's, or from approximately 0 g's to approximately 250 g's, or from approximately 0 g's to 200 g's. Moreover, the sensors may have sample frequencies adapted for modeling motions of the human body in response to impacts. Impacts to the human body such as those experienced by football players or other athletes may occur over a period of time of approximately 10 milliseconds, may include frequencies from between 0 Hz to 2000 Hz, or both. In one or more embodiments, the accelerometers, gyroscopes, and other MEMs sensing devices of the present disclosure may have sample rates ranging from approximately 10 Hz to approximately 10,000 Hz, or from approximately 500 Hz to approximately 7500 Hz, or from approximately 1000 Hz to approximately 5000 Hz, or from approximately 1500 Hz to approximately 4500 Hz, or from approximately 2500 Hz to approximately 4000 Hz, or from approximately 3000 Hz to approximately 3500 Hz, or a sample rate of approximately 3200 Hz may be used. In one or more embodiments, an accelerometer such as an ADXL 372 manufactured by Analog Devices may be provided. This particular accelerometer may have a range of 200 g's and a bandwidth or sample rate of 3200 Hz.

Apart from the kinematic sensors, the one or more sensors may include control sensors 128 adapted for use to filter data and/or control the kinematic sensors 112 to avoid collecting data, for example. In one or more embodiments, the control sensors 128 may be proximity sensors, capacitive sensors or other sensors allowing for assessments to be made about whether the mouthguard is actually in the mouth and/or on the teeth of the user. This information can be used to awaken and/or trigger the electronics of the system, to filter out data if the system is sensing information when the device is not on the teeth and/or for other purposes.

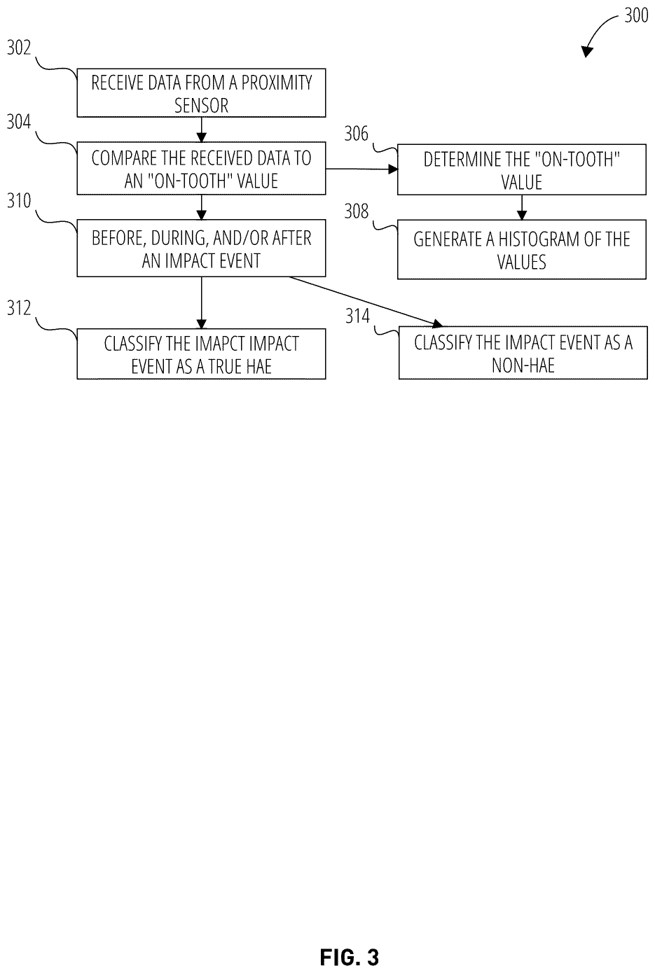

In one or more embodiments, a control sensor may be arranged facing inward from the labial portion. As such, unless an object is within the channel formed by the labial and lingual flange 120/126 and the dentition portion 118, the system may be in a sleep or off state or data collected during that timeframe may be ignored. That is, when the mouthguard is on the teeth, the sensors may be covered and an object may be sensed within the channel, so sensing with the kinematic sensors may be appropriate. However, when the mouthguard is in a case or in a gym or military bag, the sensing may not be appropriate and it may also be unlikely that the sensors would be closely covered in those situations. In some cases, users may accidentally or intentionally trigger the control sensor by obstructing the sensor. For example, a user may obstruct the sensor with their finger or the user may pop the mouthguard off of their teeth and cover the sensor with their tongue or chew on the mouthguard causing the u-shaped channel to collapse and triggering the sensor. In an example, a mouthguard may be placed in a sock, pocket or against the body in a sports-bra or jersey. In these examples, the sensor can be triggered by proximity values that can be very similar to the on-teeth and in-mouth values, which can make it desirable to further analyze data from the proximity sensor, which can include determining the on-tooth proximity values (e.g., as discussed below with respect to FIG. 3). For purposes of addressing these situations where impacts sensed during these situations may be false positives, multiple sensors have been proposed. However, the present application proposes a method of use that may lessen the need for two sensors by using analysist of a value from the sensor (e.g., a non-binary value), an orientation of the sensing device 108, or both, as discussed in more detail below. Nonetheless, multiple control sensors may still be provided for situations where users are, for example, missing teeth or have mouthguards or systems that line up with gums instead of teeth, or have other anatomical issues that make covering one sensor in a particular location difficult.

The data storage medium 114 of the electronic system may be a computer readable data storage medium such as volatile memory (e.g., random access memory (RAM)) and/or non-volatile memory (e.g., read-only memory (ROM, EPROM, EEPROM, etc.)). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data. In addition to facilitating communication and data and signals, the memory may include computer readable instructions particularly adapted for communicating with separate computing systems (e.g., for performing receiving and transmitting operations), controlling on/off states of the sensors, for monitoring the condition of the mouthguard (e.g., on the teeth, off the teeth, etc.), for receiving sensor data, for controlling on/off and/or sleep states of the processors, etc. In one or more embodiments, the memory may include computer readable instructions adapted to receive, store, and and/or analyze sensor data such as accelerometer signals received from a head impact and/or a blast event. These computer-readable instructions are discussed in more detail below.

The computer processor 116 of the electronic system may be adapted to execute the computer-readable instructions on the data storage medium. For example, the various sets of instructions on the computer readable storage medium for facilitating communication between components within the system, the more specific controls of the sensors and the receipt of data from the sensors may all be processed and/or executed by the processor. In one or more embodiments, the processor may be a high performance unit such as a 32-bit microcontroller from ST Microelectronics, for example.

Input/output devices 122 may also be present on the sensing device for powering up, for example, resetting, or otherwise directly interacting with the sensing device. Moreover, while the sensing device has been said to have computer-readable instructions and a processor for analyzing the sensor data or sensor signals, this analysis may be performed by a separate computing system as well. As such, the sensing device may be equipped with receiving and transmitting systems 124 operable by the processor and the storage medium to receive instructions from outside computing devices and/or to transmit information including the sensor data to outside computing devices. The receiving and transmitting devices may include local area network (LAN) type devices and may include WiFi, Bluetooth, Zigbee, or other relatively local area communication systems. Alternatively or additionally, the receiving and transmitting devices may include wide area network (WAN) communication capabilities such as cellular or other communication systems. As shown in FIG. 2, sensor data may be transmitted via a local area network 130, a wide area network 132 such as the internet, or via a direct hardwire communication 134 to an outside computing device 136 for monitoring and/or analysis. In one or more embodiments, a combination of these communication systems may be used.

The sensing device 108 may be the same or similar to those that are shown and described in U.S. Pat. Nos. 9,044,198, 9,149,227, 9,289,176, and 9,585,619, the contents of which are incorporated by reference herein in their entireties. Still other sensing devices and process may be used, such as those described in U.S. Pat. Nos. 8,537,017, 8,466,794, 9,526,289, 8,554,495, and 9,554,607, the contents of which are incorporated by reference herein in their entireties. Still other sensing systems and processes may be used, such as those described in U.S. patent application Ser. No. 13/009,580, 14/040,157, and 14/040,111, the contents of which are incorporated by reference herein in their entireties.

In operation and use, the sensing device 108 may be used to monitor and/or analyze impacts to athletes, military personnel, or other users. In an example, and as shown and discussed with respect to FIG. 3, a method of sensing true positive impacts (e.g., method 300) may be provided. The method may be focused on ruling out false positive sensor data allowing for a focus on true positive sensor data. In particular, the method may include a unique way of determining when a sensor is positioned on the teeth of a user during an impact. That is, when an impact sensing mouthguard is on the teeth of a user, it may be considered to be in position for sensing and impacts sensed when the mouthguard is in this position may be considered to be true positive sensor results.

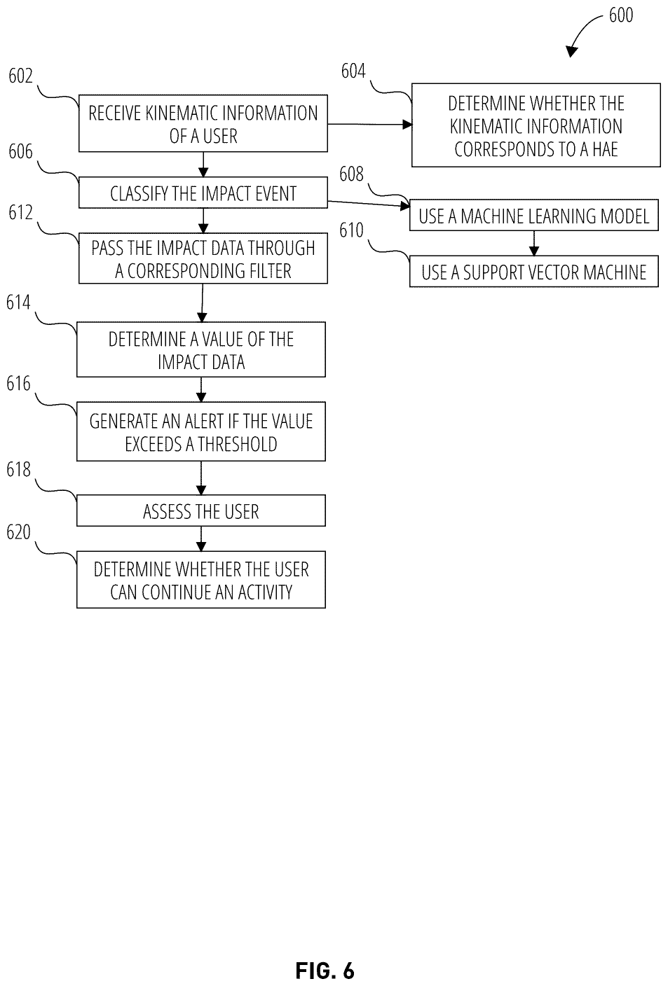

In an example, and as shown and discussed with respect to FIG. 6, a method (e.g., method 600) for classifying the impacts (e.g., the true positive impacts) may be provided. The method may be focused on classifying the impacts based on a noise level of the signal or signals corresponding to the impacts. Following the classification, the signal or signals can be filtered, for example, using a filter corresponding to the classification of the impact.

FIG. 3 shows a diagram depicting an example of portions of a method 300 for determining whether an impact sensing device (e.g., the sensing device 108) is in an engaged state (e.g., being worn by the use, being worn by the user in a way that will generate accurate data (e.g., a mouthguard being firmly on the teeth vs loosely in the mouth)). This can be important because data generated by a sensing device in an engaged state can represent true HAE data. On the other hand, data generated by a sensing device that is not in an engaged state can represent non-HAE data or data that cannot be verified to be true HAE data (e.g., because the sensing device is not engaged, the data may or may not be related to a true HAE). In the example of a wearable mouthguard, when the wearable mouthguard is engaged on the upper teeth of the user (e.g., firmly in position, such that it cannot shake or move relative to the teeth), an HAE will be transferred through the skull to the wearable mouthguard.

At step 302, data can be received from a proximity sensor (e.g., an example of the control sensors 128). The proximity sensor can be any sensor capable of generating data related to the proximity of the sensor to one or more other objects, or to determining the reflectivity of nearby objects. In an example, the proximity sensor can emit light (e.g., using an LED) and can measure an amount of the light that is reflected back to the proximity sensor, measure a total light level (e.g., reflected and ambient light), or both. The measured light value can be indicative of how close an object is to the proximity sensor (e.g., a closer object can create a more intense reflected light signal), indicative of a property of objects near the proximity sensor (e.g., more reflective objects will generate a more intense reflected light signal), or both. In an example, the closer and/or more reflective an object is, the higher the value reported by the proximity sensor. The value reported by the proximity sensor can be reported as a unitless quantity, which can include a digital value representing the ratio of the received light to the full-scale range of the light sensor. The value generated by the proximity sensor can be received by a processor (e.g., the processor 116).

At step 304, the received data (e.g., the value received in step 302) can be compared to an “on-tooth” value. The “on-tooth” value can represent a value that the proximity sensor generates when the sensing device (e.g., the sensing device 108) is in an engaged position. For example, the “on-tooth” value can represent a value that a wearable mouthguard generates when the mouthguard is firmly in place on the teeth of the user. In an example, the “on-tooth” value can be specific to a wearable device and user combination. For example, a given wearable mouthguard can have different “on-tooth” values for different users (e.g., due to different teeth positions, tooth reflectivity, etc.), a given user can have different “on-tooth” values for different wearable mouthguards (e.g., due to differences in proximity sensor or mouthguard construction, such as the thickness of plastic over the proximity sensor), or both. The on-tooth value can fluctuate day-to-day or minute-to-minute for each user, which can be due to small changes in the location and/or orientation of the sensor with respect to the tooth, which can change the true on-teeth values for the sensor. This can make it desirable to determine an “on-tooth” value for a specific wearable device and user combination. Determining the “on-tooth” value for a specific wearable device and user combination can optionally be done recurrently or periodically, which can determining an “on-tooth” value every day, every minute, or another recurrent or periodic interval.

At step 306, the “on-tooth” value can be determined. A plurality of proximity sensor measurements can be taken, and the respective values can be recorded. In an example, 2000 measurements can be made at a sampling frequency of 40 Hz. In an example, the proximity sensor measurements can be taken when it is determined that the wearable device is engaged (e.g., the user wears the wearable mouthguard during a calibration period). In an example, the proximity sensor measurements can be taken during regular use, where the wearable device may or may not be engaged. The calibration period may or may not require any input and/or effort by the user. In an example, it can occur in the background automatically. In an example, it can be initiated by personnel who are responsible for collecting and/or monitoring head impact data for the user. The plurality of values can be processed to determine an “on-tooth” value or “on-tooth” range of values. For example, a central tendency (e.g., mean, median, or mode) or moving average of the values can be determined, such as in a case where it is determined that the wearable device is engaged during the measurement of the values. In an example, additional processing can be done, which can include removing values below a specified threshold, values above a specified threshold, or both, before determining a central tendency or moving average.

At step 308, a histogram of the values can be generated (e.g., populated). The histogram can optionally be normalized. A peak in the histogram (e.g., a location where a specified proximity sensor value stands out as compared to neighboring value bins) with the highest proximity sensor value can be recorded. For example, the largest peak above a specified threshold level (e.g., 200 prox units) can be selected and recorded. This value can be determined as the “on-tooth” value. Recording the largest commonly occurring value can be likely to provide an accurate “on-tooth” value because the teeth are the closest and most reflective objects that the sensor is likely to be around consistently during use. For example, other more reflective objects may be near the proximity sensor during use, but not as long and/or as often as the teeth. The use of the histogram can help to remove spurious signals or outliers.

In an example, step 306 and step 308 can be repeated, which can generate a peak histogram value for each iteration of step 306 and step 308. The respective values from each iteration can be processed (e.g., to determine a central tendency), which can include using a median filter, such as a 5-point median filter. Step 306 and step 308 can be performed recurrently (e.g., periodically), which can include being performed recurrently during runtime. For example, the wearable device can perform operations to determine the “on-tooth” value at specified intervals or substantially continuously. This can help allow the “on-tooth” value to remain accurate throughout the use of the device.

Optionally, at step 306, one or more “off-teeth” values can be determined. In an example, the proximity sensor value can be recorded while the wearable device is charging. For example, a plurality of measurements can be taken using the proximity sensor while the wearable device is charging, and the measured values can be processed (e.g., such as to determine a central tendency) to determine an “open-air” value for the wearable device. This operation can be performed at run-time (e.g., after the wearable device has left the factory) to account for differences between devices or changes in the device over time. The “on-tooth” value used at step 304 can be determined in any way, and is not limited to being determined as discussed with respect to step 306 and step 308.

In an example, an on-tooth value for a wearable mouthguard can generally range from 350-1000 prox units. In open-air (e.g., when the wearable mouthguard is sitting on a desk) the value can generally be 50-100 prox units. In a football helmet facemask the value can generally be 1500-2500 prox units (e.g., due to the metal facemask being reflective). In a sock or a pocket the value can generally be 300-600 prox units. In some cases, there can be overlap between the “on-tooth” value and conditions in which the proximity sensor is not on-tooth (e.g., the sock or pocket value can be close to the “on-tooth” value). This can make it desirable to include other steps in addition to comparing the received data to the “on-tooth” value.

At step 310, the data received from the proximity sensor can be compared to the “on-tooth” value before, during, and/or after an impact event. For example, the proximity sensor can generate a stream of values, such as at a recurrent interval (e.g., 100 Hz). This stream of values can be recorded or persisted, at least for a given number of values (e.g., the most recent 1000 values are recorded) or length of time (e.g., the last 5 seconds of data are recorded). Following the determination that there has been an impact event (e.g., due to an accelerometer or gyro value crossing a specified threshold), the recorded proximity sensor values before, during, and/or after the impact event can be accessed. These recorded proximity sensor values can be used to determine whether the event is a true HAE.

At step 312, the impact event can be classified as a true HAE. In an example, if the proximity sensor value before and after the impact are within a specified first threshold range of the “on-tooth” value, the impact event can be classified as a true HAE, which can include being classified as a tier 0 true HAE. For example, if the “on-tooth” value is 400 prox units and the specified first threshold range is 20 prox units, if the before impact and the after impact values are between 380 and 420 prox units, the event can be classified as a true HAE. The time length between the impact event and the before and after impact times can be set to any value. In an example, the before impact value is measured 0.5 seconds before the impact event and the after impact value is measured between 0.5 and 2 seconds after the impact event.

In an example, alternatively or in addition to the example above (e.g., the tier 0 true HAE), if the value before impact or after impact is within the specified first threshold range of the “on-tooth” value and the other of the before or after impact value is within a specified second threshold range of the value within the specified first threshold range of the “on-tooth” value, the impact event can be classified as a true HAE, which can include being classified as a tier 1 true HAE. In an example, the specified second threshold range can be 40 prox units. In this example, if either of the before or after impact values are between 380 and 420 prox units, and the other value is within 40 prox units of that value, the event can be classified as a true HAE.

In an example, alternatively or in addition to the examples above (e.g., the tier 0 and tier 1 true HAEs), if the value before impact or after impact is within the specified first threshold range of the “on-tooth” value and the other of the before or after impact value is outside of the specified second threshold range but within a specified third threshold range of the value within the specified first threshold range of the “on-tooth” value, the impact event can be classified as a true HAE, which can include being classified as a tier 2 true HAE. In an example, the specified third threshold range can be 100 prox units. In this example, if either of the before or after impact values are between 380 and 420 prox units, and the other value is outside of 40 prox units but within 100 prox units of that value, the event can be classified as a true HAE. Impact events classified into different tiers can be used and or processed differently. In an example, only tier 0 impact events can be treated as true HAEs.

At step 314, the impact event can be classified as a non-HAE. For example, if the event is not classified as a true HAE in one or more of the above examples (e.g., tier 0, tier 1, or tier 2), the event can be classified as a non-HAE.

In an example, the method 300 can include considering the orientation of the sensing device, alternatively or in addition to the proximity data. For example, an orientation of the sensing device can be determined using an orientation sensor, the gravity vector determined by a 3-axis accelerometer, a gyro, a tilt sensor, or a magnetometer. The orientation data can be used in determining the “on-tooth” value. For example, values that are collected when the sensing device is not within a specified angular threshold from vertical (e.g., 10 degrees, 20 degrees) can be discarded (e.g., not used to form the histogram in step 308). This can filter out one or more conditions where the sensing device is not engaged. Because the determination of the “on-tooth” value is based on a plurality of proximity sensor values, discarding one or more values that represent good “on-tooth” values because the sensing device is not in an upright orientation may not negatively affect the determination of the “on-tooth” value, or the benefits of discarding one or more bad values can outweigh a negative effect of discarding good values.

The orientation of the sensing device can be used to enable or disable sensor features (e.g., to conserve power). For example, a user may generally be in an upright position when the sensing device is first engaged (e.g., a user is generally upright when placing a mouthguard). One or more features can be enabled when the sensing device transitions to an upright orientation.

Optionally, the orientation information can be used to determine whether an impact event is a true HAE. For example, the orientation of the sensing device before, during, or after the impact event can be considered in classifying the impact event as a true HAE or a non-HAE. In an example, an impact event will be classified as a true HAE if the orientation of the sensing device indicates that the device was substantially upright within a threshold time (e.g., 3 seconds) of the impact event (e.g., due to a player standing substantially upright with an engaged device prior to a play or impact). In an example, the orientation information may only be considered when the wearable device is motionless (e.g., while a player is getting set before a play). For example, if the “on-tooth” value indicates that the wearable device is engaged, the wearable device is motionless, and the wearable device is upright, any following impact can be determined as a true HAE. Vertical and/or upright can indicate a position the wearable device is in when the user is standing upright. For example, a wearable mouthguard can be upright when engaged on the user and the users head is in a generally neutral position looking horizontally.

The shown order of steps is not intended to be a limitation on the order in which the steps are performed. In an example, two or more steps may be performed simultaneously or at least partially concurrently.

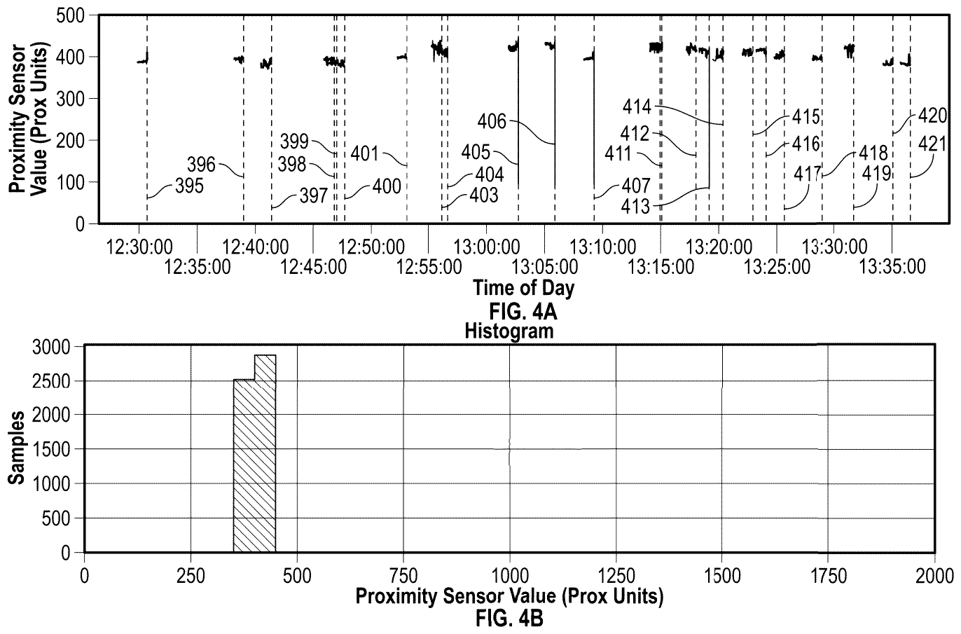

FIG. 4A shows an example of on-person collected (e.g., field collected from a user playing in a game) proximity sensor values over time. FIG. 4B shows an example of a histogram of the collected proximity sensor values of FIG. 4A. FIG. 4A and FIG. 4B, which can show an example of performing portions of the method 300, will be discussed together below. FIG. 4A shows a number of impact events including impact event 395 to impact event 421. FIG. 4A shows that the proximity sensor may only generate values under specified conditions, which can include when the athlete is moving or when the wearable device can detect that a user is nearby (e.g., when a wearable mouthguard determines that it is inside the mouth of a user). The values in FIG. 4A can be measured and/or recorded at step 306. FIG. 4A shows that the most commonly occurring value is approximately 380-390 prox units. This value was collected at times when the wearable mouthguard was engaged. FIG. 4B shows a significant peak in the bins ranging from approximately from 340 to 420 prox units, which can correspond to the “on-tooth” value shown in FIG. 4A of 380-390 prox units. The histogram in FIG. 4B can be generated at step 308. In this example, the histogram bins are approximately 50 prox units in range, but any size of bin can be used. In this example, the off-teeth “open-air” value was about 85 prox units, as can be seen in FIG. 4A around time 13:05.00 (e.g., the prox value quickly drops from about 390 to 95, which can indicate the wearable mouthguard was removed from the teeth). The most populous bin (e.g., the bin ranging from about 400-450 prox units) can be selected (e.g., at step 308) as the “on-tooth” value or selected for use in further operations (e.g., a moving average of most populous histogram bins).

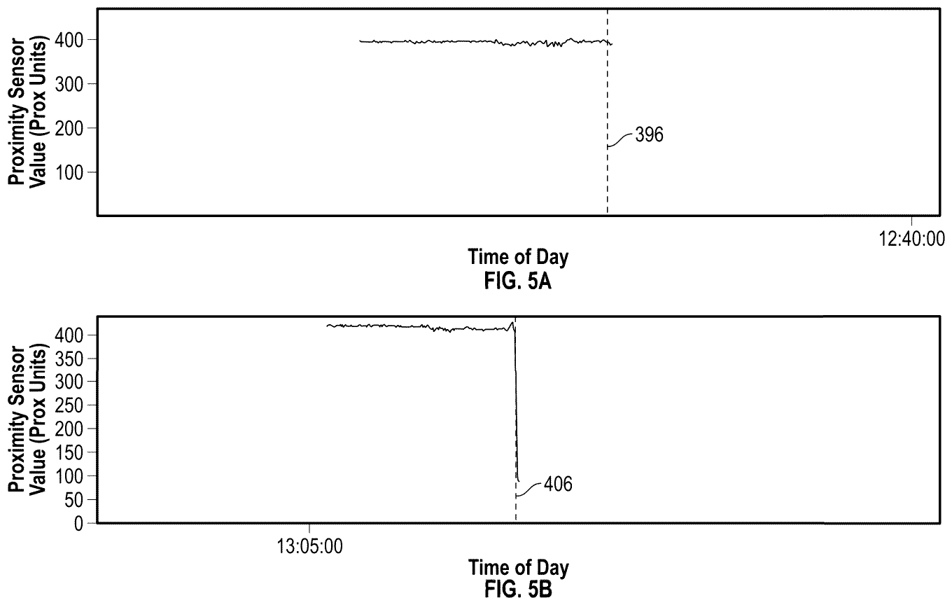

FIG. 5A shows an example of the proximity sensor values of FIG. 4A near impact event 396. FIG. 5A shows that the proximity sensor values were near the “on-tooth” value before and after the impact event. This can be determined at step 304 and step 310, and can result in classifying impact event 396 as a true HAE at step 312.

FIG. 5B shows an example of the proximity sensor values of FIG. 4A near an impact event 406. FIG. 5B shows that the proximity sensor values were near the “on-tooth” value before the impact event, but were significantly below the “on-tooth” value after the impact event (e.g., near the “open-air” value). This can be determined at step 304 and step 310, and can result in classifying impact event 406 as a non-HAE at step 312. FIG. 5B can correspond to a user removing the wearable mouthguard, which can trigger an impact event, but can also include a change in the proximity sensor value.

FIG. 6 shows a diagram depicting an example of a method 600 for classifying impact data (e.g., data corresponding to an HAE). The method 600 can be performed alternatively or in addition to one or more portions of the method 300. In an example, the method 600 can be performed after the method 300 (e.g., after the impact event is classified as a true HAE at step 312). The method 600, the method 300, or both can be performed on a monitoring device system, which can include one or more wearable devices (e.g., a wearable mouthguard, such as the sensing device 108) and one or more non-wearable devices (e.g., computers, networks, servers, as shown and discussed with respect to FIG. 2). The method 600 and/or method 300 can be performed in a distributed fashion, with some devices performing some steps or portions of steps and other devices performing other steps or portions of steps.

At step 602, kinematic information of a user can be received, which can include using a signal receiver circuit. The kinematic information can be measured using a wearable device, and the signal receiver circuit can be included on the wearable device, another device, or partially included on both. The kinematic information can include information from one or more sensors (e.g., the sensors 112), which can include accelerometers, gyroscopes, or other sensors capable of generating a signal indicative of the kinematic motion of a user. The kinematic information can include impact data, corresponding to an impact event. The impact data can correspond to a head acceleration event (HAE) (e.g., a true HAE), or can correspond to a non-HAE. The impact data can include data from one or more sensors corresponding to the time during, before, and/or after the impact event (e.g., data showing the full impact event, which can include the ramp up from baseline to maximum levels and the ramp down from maximum levels to baseline). In an example, the impact data includes a time series of one or more of acceleration values (e.g., linear and/or angular acceleration in between 1 to 3 axes) or velocity values (linear and/or angular velocity in between 1 to 3 axes) from a specified range spanning between 30 milliseconds before a peak of an impact event to 30 milliseconds after the peak of an impact event.

At step 604, it can optionally be determined whether the kinematic information corresponds to a HAE. In an example, one or more portions of the method 300 are performed at step 604 to determine whether an impact event is a true HAE, and the impact data is only passed to the method 600 when it is determined that the impact data corresponds to a true HAE.

At step 606, the impact event can be classified (e.g., classified based on a noise level, such as following determining that the impact event is a true HAE in step 604), which can include using an assessment circuit. The impact event can be classified based on one or more features indicative of a noise level of the impact data. The noise level can refer to the presence of any signals in the impact data that are not indicative of the head motion of the user, which can include one or more of measurement noise, circuit noise, or signals that are indicative of the wearable device moving independent of the head of the user (e.g., a wearable mouthguard moving or vibrating on the teeth, which can produce signals indicative of mouthguard motion that are not indicative of head motion, a wearable mouthguard being directly struck (e.g., the wearer was hit in the mouth), which can produce signals indicative of the mouthguard moving with a greater magnitude than the magnitude of movement passed to the head from the hit (e.g., due to mouthguard vibration or flex, or the flex of the upper jaw relative to the center of gravity of the head)). For example, any unwanted signal can be referred to generally as “noise.”

The one or more features can include features generated by a computer program or features generated by a human operator. For example, someone skilled in head acceleration event sensing may look at a plurality of data sets and pick out features that are indicative of clean signals vs noisy signals. The one or more features can include any number of features, such as 10 features, 20 features, or 50 features. In an example, one or the features includes a ratio of the translational, tangential, and centripetal acceleration to a total peak linear acceleration of a center of gravity of a head of the user. The one or more features indicative of a noise level can include one or more features indicative of a signal-to-noise ratio (e.g., a ratio of the wanted signal to the unwanted signal).

The impact data can be classified into one or more classifications, which can include one classification, two classifications, three classifications, four classifications, or five or more classifications. The classifications can differ based on the determined or estimated noise level (e.g., signal-to-noise ratio) of the impact data. In an example, there can be two classifications, which can include a low noise level classification and a high noise level classification. The impact data in the low noise level classification can be determined to have a lower noise level than impact data in the high noise level classification. In an example, there can be three classifications, which can include a low noise level classification, a medium noise level classification, and a high noise level classification. The impact data in the low noise level classification can be determined to have a lower noise level than impact data in the medium noise level classification, which can be determined to have a lower noise level than impact data in the high noise level classification.

At step 608, classifying the impact event based on one or more features indicative of a noise level can include using a machine learning model to classify the impact event. For example, the machine learning model can be trained using datasets including impact events that have been classified based on noise level (e.g., classified HAEs, which can include impact events that were determined to be true HAEs and were then classified based on noise level). These classified impact events can be classified by a human operator (e.g., viewing impact data and determining a relative noise level of the impact data), by laboratory testing (e.g., comparing the actual workload of an impact to the workload indicated by the kinematic information), or both. For example, a plurality of impact events can be reviewed and classified based on their noise level, such as by a human operator. The plurality of impact events can correspond to true HAEs (e.g., the impact data is determined to be a true HAE before being included in the classified impact events for training). The machine learning model can be trained prior to runtime (e.g., before a sporting event), during runtime, or both (e.g., adaptive machine learning). The machine learning model can run on the wearable device (e.g., wearable mouthguard), a remote computer (e.g., on the sidelines, in the cloud), or both.

At step 610, using a machine learning model can include using a support vector machine (SVM). The SVM can be or have been trained to classify impact events based on their noise level, which can include using the one or more features indicative of a noise level of the impact event. As discussed above, one or more of the features can be passed to the SVM (e.g., the SVM can be “instructed” on features to look for), and/or one or more features can be generated by the SVM (e.g., the SVM can determine features based on the training data set). In an example, using a machine learning model can include using a neural network model, alternatively or in addition to an SVM. The neural network can be configured and/or trained in any fashion, which can include being trained to classify impact events based on their noise level.

In an example, as discussed above, before being classified by the machine learning model, it can be determined that the impact data corresponds to an HAE. For example, rather than using the machine learning model to determine if the impact data corresponds to a HAE, the machine learning model can be used to classify events that have been determined as true HAEs. In an example, a machine learning model can be used to determine if the impact event is a true HAE (e.g., by looking at the kinematic information, by looking at other information, such as in the method 300 (e.g., to analyze proximity data)), and a separate machine learning model can be used to classify the impact event based on noise level (e.g., the machine learning model discussed with respect to steps 608 and 610). In an example, no machine learning model is employed in classifying the impact even as a true HAE, and the machine learning model discussed with respect to steps 608 and 610 (e.g., for noise level) is the first machine learning model used on the impact data. In an example, other machine learning models can be used on or in correlation with the system, but the machine learning model discussed with respect to steps 608 and 610 is the first machine learning model used on the kinematic information (e.g., a machine learning model may be used on the proximity data).

At step 612, the impact data can be passed through a corresponding filter to generate filtered impact data, where the corresponding filter can be based upon the classification performed in step 606. The filter can include a highpass filter, a lowpass filter, or a bandpass filter. In an example, for some classifications, the filtered impact data may match the impact data, and the corresponding filter can include one or more of no filter or a simple pass through device.

In an example, to pass the impact data through a corresponding filter can include passing the impact data through a lowpass filter. A corner frequency of the lowpass filter can be relatively higher for impact data classified as having a relatively lower signal-to-noise ratio. In an example with a low noise level classification and a high noise level classification, impact data in the low noise level classification can be passed through a lowpass filter with a corner frequency of between 160 hertz (Hz) and 200 Hz inclusive. Impact data in the high noise level classification can be passed through a lowpass filter with a corner frequency of between 50 Hz and 120 Hz inclusive.

In an example with a low noise level classification, a medium noise level classification, and a high noise level classification, impact data in the low noise level classification can be passed through a lowpass filter with a corner frequency of 200 Hz. Impact data in the medium noise level classification can be passed through a lowpass filter with a corner frequency of 100 Hz. Impact data in the high noise level classification can be passed through a lowpass filter with a corner frequency of 50 Hz.

In an example, passing the impact data through a corresponding filter at step 614 to generate filtered impact data can include adjusting the filtered impact data for an estimated or specified attenuation caused by the filter. For example, an specified (e.g., estimated, measured, or calibrated) attenuation can be determined, and the filtered impact data can be amplified to account for this attenuation (e.g., an amplification to recover the attenuated signal). In an example, filtered impact data from the low noise level (e.g., 200 Hz corner frequency) can include a limited attenuation, and might not be corrected for. Filtered impact data from the medium noise level (e.g., 100 Hz corner frequency) can include a 2 percent attenuation in peak linear velocity and a 15 percent attenuation in peak linear acceleration. One or more of these attenuations can be adjusted or corrected for. Filtered impact data from the high noise level (e.g., 50 Hz corner frequency) can include a 4 percent attenuation in peak linear velocity and a 43 percent attenuation in peak linear acceleration. One or more of these attenuations can be adjusted or corrected for. Other values can experience attenuation, and can be adjusted or corrected for.

In an example, a lookup table can be used to determine an adjustment factor for one or more values. Table 1 shows an example of adjustment values for peak linear acceleration (PLA), peak angular acceleration (PAA), work (e.g., workload), peak linear velocity (PLV), and peak angular velocity (PAV). The filtered value can be multiplied by the adjustment value to adjust for a signal attenuation. Class 0 can correspond to a 200 Hz corner frequency lowpass filter, Class 1 can correspond to a 100 Hz corner frequency lowpass filter, and Class 2 can correspond to a 50 Hz corner frequency lowpass filter. The values in table 1 can include values derived from empirical data from crash test dummy experiments at Virginia Tech University.

TABLE 1
Lookup Table for PLA, PAA, Work, PLV, PAV

| | Class 0 | Class 1 | Class 2 |
|---|---|---|---|
| PLA | 1.0 | 1.3 | 2.0 |
| PAA | 1.0 | 1.3 | 2.0 |
| Work | 1.0 | 1.1 | 2.8 |
| PLV | 1.0 | 1.1 | 1.9 |
| PAV | 1.0 | 1.0 | 1.1 |

At step 614, a value of the HAE can be determined by analyzing the filtered impact data, which can include using an assessment circuit. The value an include one or more of a peak linear acceleration (e.g., of the head of a user), a peak angular acceleration (e.g., of the head of a user), a peak angular velocity (e.g., of the head of a user), a peak linear velocity (e.g., of the head of a user), a workload (e.g., of the head of a user), an impact direction (e.g., of the head of a user), or an impact location (e.g., of the head of a user).

At step 616, an alert can be generated if a value exceeds a threshold. For example, thresholds can be set on one or more values such that, when exceeded, the user must be further examined, discontinue the activity, or both. For example, if a total workload (e.g., across multiple impacts in one game) for a football player exceeds a threshold, or if a value (e.g., peak linear velocity, peak angular velocity, a composite value, which can include both peak angular velocity and peak linear velocity, etc.) exceeds a specified threshold, an alert can be generated, such as at step 616, to further examine the football player (e.g., assess for a concussion) or remove the football player from gameplay. The alert can be transmitted from the wearable device to a remote system (e.g., a system on the sidelines), or can be generated by the remote system (e.g., the remote system is analyzing data and generating alerts).

At step 618, the user can be assessed, which can be done in response to the alert generated at step 616. This can include temporarily or permanently removing the user from game play. For example, staff can receive the alert and flag the user for assessment. In an example, there may not be other indications (e.g., visual indications or indications from the user) that an assessment is necessary.

At step 620, it can be determined whether the user can continue an activity. For example, it can be determined whether an athlete can continue participation in a sporting event. For example, staff on the sidelines during a game may assess the user (e.g., using a concussion protocol), and determine whether the user can continue the activity. This can be determined at least in part using one or more values determined in step 614.

In an example, the monitoring device system includes a wearable mouthguard. To determine that the impact data corresponds to an HAE, the signal receiver circuit can be configured to receive proximity data (e.g., at step 302) from a proximity sensor in the wearable mouthguard and the assessment circuit can be configured to process proximity data (e.g., in the method 300) from the wearable mouthguard, which can determine whether the wearable mouthguard is engaged on the teeth of the user. To determine if the wearable mouthguard is engaged on the teeth of the user can include comparing proximity data received before and after the impact data to a threshold range of values (e.g., at steps 304 and 310).

In an example, to determine that the impact data corresponds to an HAE can include receiving (e.g., using the signal receiver circuit) orientation data related to an orientation of the wearable mouthguard, such as discussed above. The method 600 can include determining (e.g., using the assessment circuit), whether the orientation data indicates that the wearable mouthguard is in a generally upright orientation (e.g., upright within a specified threshold range of vertical).

In an example, the signal receiver circuit, the assessment circuit, or both, are included in a wearable mouthguard. The wearable mouthguard can also include a communication circuit, which can be configured to transmit a representation of the filtered impact data (e.g., transmit the filtered impact data for processing by another system).

The shown order of steps is not intended to be a limitation on the order in which the steps are performed. In an example, two or more steps may be performed simultaneously or at least partially concurrently.

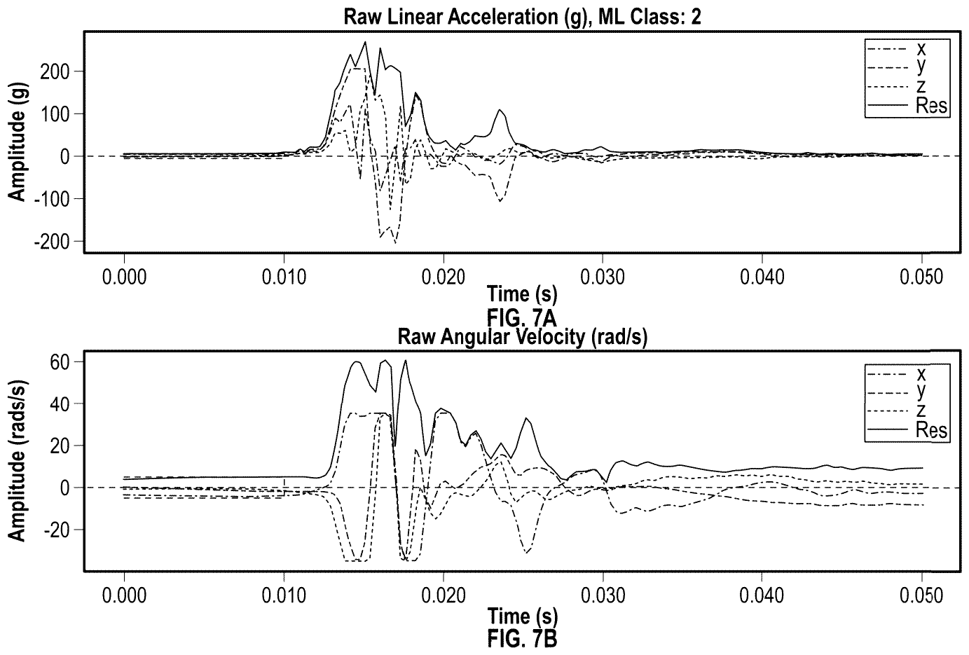

FIG. 7A and FIG. 7B show data from a real impact event measured using a wearable mouthguard. FIG. 7A shows an example of unfiltered linear acceleration values. FIG. 7B shows an example of unfiltered angular velocity values. The impact event of FIG. 7A and FIG. 7B can represent a Class 2 (e.g., high noise level) true HAE. The linear acceleration and angular velocity can include values in the x, y, and z directions, as well as a resulting composite value (e.g., res, which can represent the magnitude of the combined, x, y, and z axes (e.g., the resultant vector magnitude)).

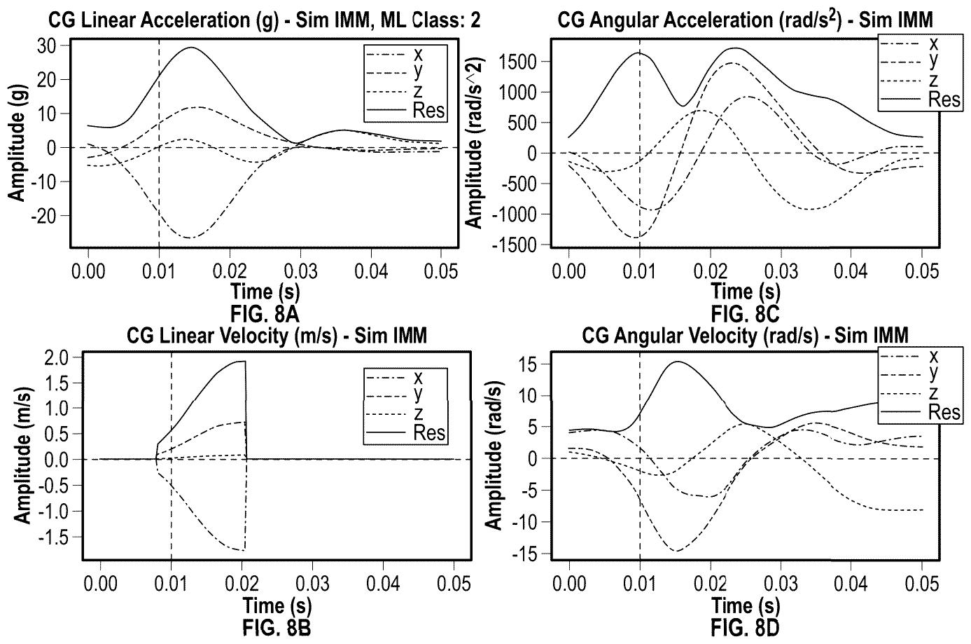

FIG. 8A through FIG. 8D show time plots of data that was generated from the data of FIG. 7A and FIG. 7B. In FIG. 8A through FIG. 8D, the data from FIG. 7A and FIG. 7B was processed to determine values experienced by the center of gravity of the users head. These data sets were also filtered with a 50 Hz filter.

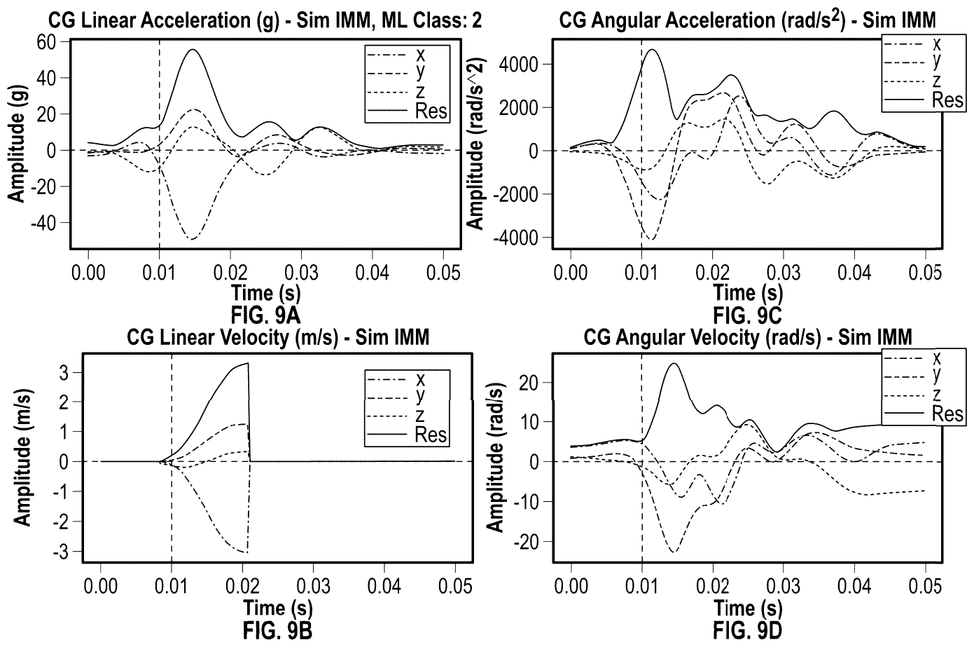

FIG. 9A through FIG. 9D show time plots of data that was generated from the data of FIG. 7A and FIG. 7B. In FIG. 9A through FIG. 9D, the data from FIG. 7A and FIG. 7B was processed to determine values experienced by the center of gravity of the users head. These data sets were also filtered with a 100 Hz filter.

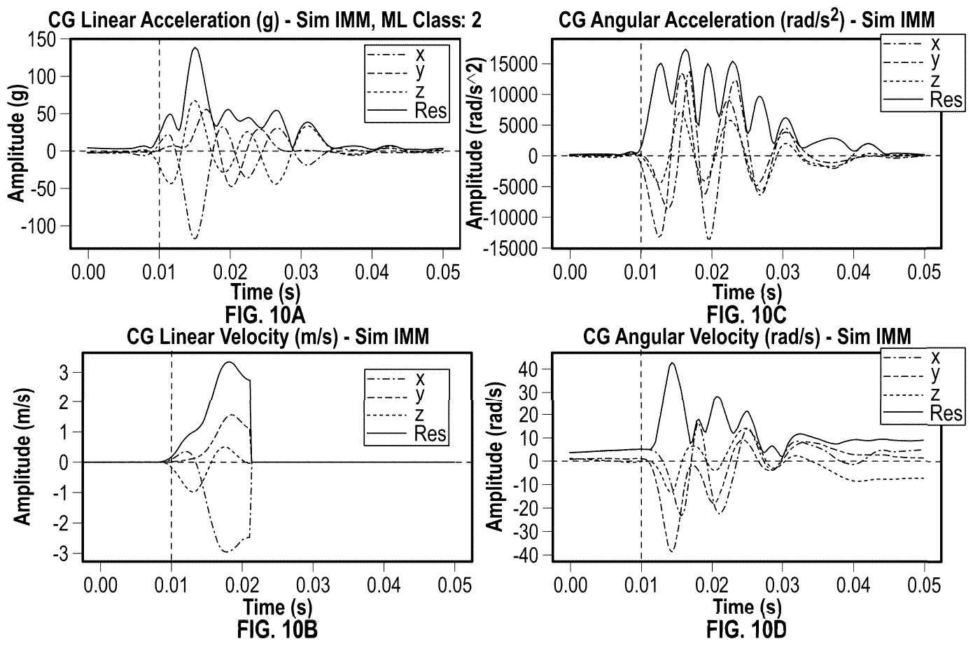

FIG. 10A through FIG. 10D show time plots of data that was generated from the data of FIG. 7A and FIG. 7B. In FIG. 10A through FIG. 10D, the data from FIG. 7A and FIG. 7B was processed to determine values experienced by the center of gravity of the users head. These data sets were also filtered with a 200 Hz filter. FIG. 8A through FIG. 10D show that the amplitude of the plots can be larger in plots that were filtered with a filter having a larger corner frequency. FIG. 8A through FIG. 10D show that the plots filtered with a filter having a smaller corner frequency can be smoother.

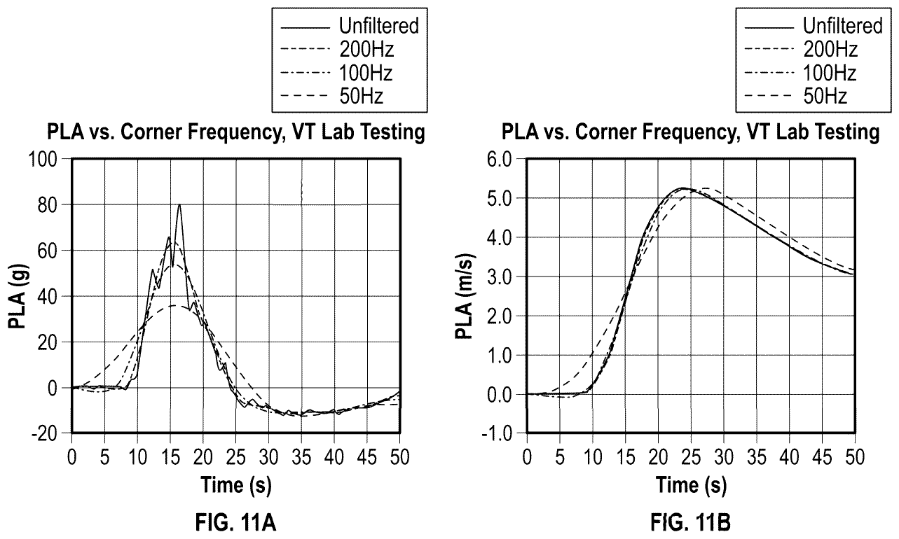

FIG. 11A shows an example of filtering peak linear acceleration. FIG. 11B shows an example of filtering peak linear velocity. FIG. 11A and FIG. 11B show plots of PLA and PLV over time, respectively, for unfiltered data, data filtered with a 200 Hz corner frequency lowpass filter, data filtered with a 100 Hz corner frequency lowpass filter, and data filtered with a 50 Hz corner frequency lowpass filter. FIG. 11A shows that the PLA can experience a significant attenuation from unfiltered data at lower corner frequencies. This signal attenuation can be due to the removal of higher frequency signals, which can represent harmonics (e.g., higher order harmonics of head vibration) or noise. FIG. 11B shows that the PLV can be substantially consistent across a range of corner frequencies as compared to unfiltered data.

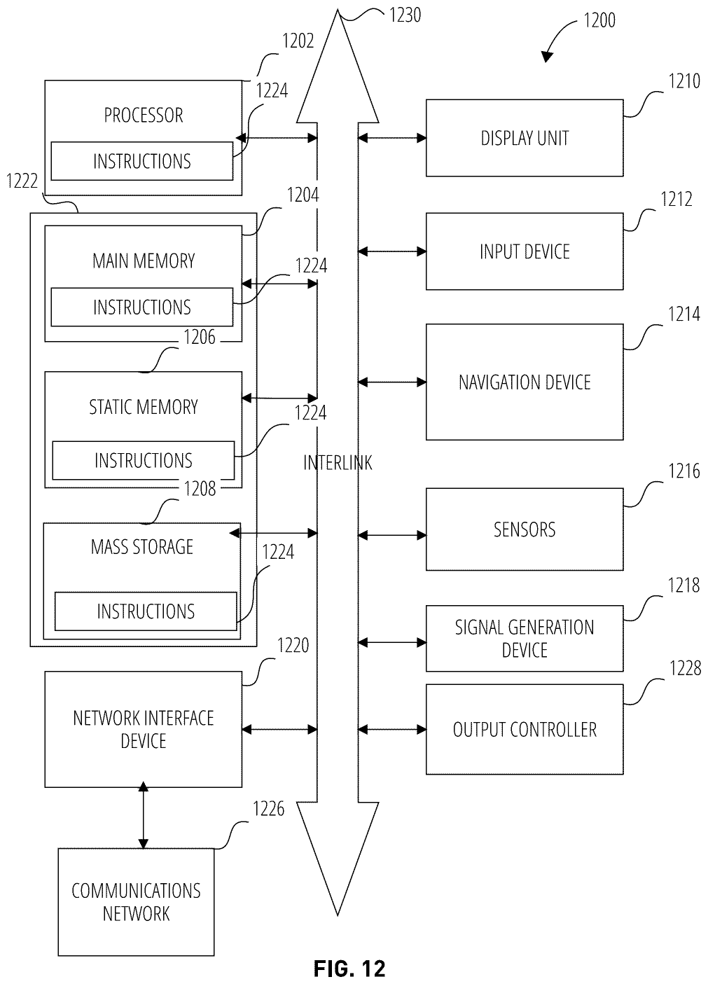

FIG. 12 illustrates a block diagram of an example machine 1200 upon which any one or more of the techniques (e.g., methodologies) discussed herein may be implemented. Examples, as described herein, may include, or may operate by, logic or a number of components, or mechanisms in the machine 1200. Circuitry (e.g., processing circuitry) is a collection of circuits implemented in tangible entities of the machine 1200 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1200 follow.

In alternative examples, the machine 1200 may operate as a standalone device or may be connected (e.g., networked) to other machines. In a networked deployment, the machine 1200 may operate in the capacity of a server machine, a client machine, or both in server-client network environments. In an example, the machine 1200 may act as a peer machine in peer-to-peer (P2P) (or other distributed) network environment. The machine 1200 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1200 may include a hardware processor 1202 (e.g., a central processing unit (CPU), a graphics processing unit (GPU), a hardware processor core, or any combination thereof), a main memory 1204, a static memory (e.g., memory or storage for firmware, microcode, a basic-input-output (BIOS), and mass storage 1208 (e.g., hard drives, tape drives, flash storage, or other block devices) some or all of which may communicate with each other via an interlink 1230 (e.g., bus). The machine 1200 may further include a display unit 1210, an alphanumeric input device 1212 (e.g., a keyboard), and a user interface (UI) navigation device 1214 (e.g., a mouse). In an example, the display unit 1210, input device 1212 and UI navigation device 1214 may be a touch screen display. The machine 1200 may additionally include a signal generation device 1218 (e.g., a speaker), a network interface device 1220, and one or more sensors 1216, such as a global positioning system (GPS) sensor, compass, accelerometer, or other sensor. The machine 1200 may include an output controller 1228, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1202, the main memory 1204, the static memory 1206, or the mass storage 1208 may be, or include, a machine readable medium 1222 on which is stored one or more sets of impact data structures or instructions 1224 (e.g., software) embodying or utilized by any one or more of the techniques or functions described herein. The instructions 1224 may also reside, completely or at least partially, within any of registers of the processor 1202, the main memory 1204, the static memory 1206, or the mass storage 1208 during execution thereof by the machine 1200. In an example, one or any combination of the hardware processor 1202, the main memory 1204, the static memory 1206, or the mass storage 1208 may constitute the machine readable media 1222. While the machine readable medium 1222 is illustrated as a single medium, the term “machine readable medium” may include a single medium or multiple media (e.g., a centralized or distributed database, and/or associated caches and servers) configured to store the one or more instructions 1224.

The term “machine readable medium” may include any medium that is capable of storing, encoding, or carrying instructions for execution by the machine 1200 and that cause the machine 1200 to perform any one or more of the techniques of the present disclosure, or that is capable of storing, encoding or carrying impact data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 1222 may be representative of the instructions 1224, such as instructions 1224 themselves or a format from which the instructions 1224 may be derived. This format from which the instructions 1224 may be derived may include source code, encoded instructions (e.g., in compressed or encrypted form), packaged instructions (e.g., split into multiple packages), or the like. The information representative of the instructions 1224 in the machine readable medium 1222 may be processed by processing circuitry into the instructions to implement any of the operations discussed herein. For example, deriving the instructions 1224 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 1224.

In an example, the derivation of the instructions 1224 may include assembly, compilation, or interpretation of the information (e.g., by the processing circuitry) to create the instructions 1224 from some intermediate or preprocessed format provided by the machine readable medium 1222. The information, when provided in multiple parts, may be combined, unpacked, and modified to create the instructions 1224. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 1224 may be further transmitted or received over a communications network 1226 using a transmission medium via the network interface device 1220 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet impact data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless impact data networks (e.g., Institute of Electrical and Electronics Engineers (IEEE) 802.11 family of standards known as Wi-Fi®, IEEE 802.15.4 family of standards, peer-to-peer (P2P) networks, among others. In an example, the network interface device 1220 may include one or more physical jacks (e.g., Ethernet, coaxial, or phone jacks) or one or more antennas to connect to the communications network 1226. In an example, the network interface device 1220 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1200, and includes digital or analog communications signals or other intangible medium to facilitate communication of such software. A transmission medium is a machine-readable medium.

The following, non-limiting examples, detail certain aspects of the present subject matter to solve the challenges and provide the benefits discussed herein, among others.

### EXAMPLES

Example 1 is a monitoring device system, the system comprising: a signal receiver circuit configured to receive kinematic information of a user, the kinematic information including impact data corresponding to a head acceleration event (HAE); and an assessment circuit configured to process the impact data, including to: classify the HAE based on one or more features indicative of a noise level of the HAE; and based upon the classification, pass the impact data through a corresponding filter to generate filtered impact data.

In Example 2, the subject matter of Example 1 optionally includes wherein the assessment circuit is further configured to determine a value of the HAE by analyzing the filtered impact data.

In Example 3, the subject matter of Example 2 optionally includes wherein the assessment circuit is further configured to compare the value to a threshold, and, when the value is on a specified side of the threshold, trigger an alert to be generated.

In Example 4, the subject matter of any one or more of Examples 2-3 optionally include wherein the value includes at least one of peak linear acceleration, peak angular acceleration, peak angular velocity, peak linear velocity, workload, impact direction, or impact location.

In Example 5, the subject matter of any one or more of Examples 2-4 optionally include wherein the value of the HAE is used to determine whether the user can continue participation in a sporting event.

In Example 6, the subject matter of any one or more of Examples 1-5 optionally include wherein to classify the HAE based on one or more features indicative of a noise level includes using a machine learning model to classify the HAE.

In Example 7, the subject matter of Example 6 optionally includes wherein the machine learning model has been trained using datasets including classified HAEs.

In Example 8, the subject matter of Example 7 optionally includes wherein the machine learning model includes a support vector machine (SVM), wherein the SVM has been trained to classify HAEs based on their noise level using the one or more features indicative of a noise level of the HAE.

In Example 9, the subject matter of Example 8 optionally includes wherein the one or more features include a plurality of features, wherein one or the plurality of features includes a ratio of the translational, tangential, and centripetal acceleration to a total peak linear acceleration of a center of gravity of a head of the user.

In Example 10, the subject matter of any one or more of Examples 6-9 optionally include wherein the machine learning model includes a neural network model, wherein the neural network model has been trained to classify HAEs based on their noise level.

In Example 11, the subject matter of any one or more of Examples 6-10 optionally include wherein, before being classified by the machine learning model, it is determined that the impact data corresponds to an HAE.

In Example 12, the subject matter of Example 11 optionally includes wherein the monitoring device system includes a wearable mouthguard, wherein to determine that the impact data corresponds to an HAE, the signal receiver circuit is configured to receive proximity data from a proximity sensor in the wearable mouthguard and the assessment circuit is configured to process proximity data from the wearable mouthguard and determine whether the wearable mouthguard is engaged on the teeth of the user.

In Example 13, the subject matter of Example 12 optionally includes wherein to determine if the wearable mouthguard is engaged on the teeth of the user includes comparing proximity data received before and after the impact data to a threshold range of values.

In Example 14, the subject matter of any one or more of Examples 12-13 optionally include wherein to determine that the impact data corresponds to an HAE further includes: receiving, using the signal receiver circuit, orientation data related to an orientation of the wearable mouthguard; and determining, using the assessment circuit, whether the orientation data indicates that the wearable mouthguard is in a generally upright orientation.

In Example 15, the subject matter of any one or more of Examples 1-14 optionally include wherein to pass the impact data through a corresponding filter comprises passing the impact data through a lowpass filter.

In Example 16, the subject matter of Example 15 optionally includes wherein a corner frequency of the lowpass filter is higher for HAEs classified as having a lower noise level.

In Example 17, the subject matter of Example 16 optionally includes wherein: the classifications include a low noise level classification and a high noise level classification; impact data in the low noise level classification are determined to have a lower noise level than impact data in the high noise level classification; impact data in the low noise level classification is passed through a lowpass filter with a corner frequency of between 17 is missing parent: 160 hertz and 17 is missing parent: 200 hertz inclusive; and impact data in the high noise level classification is passed through a lowpass filter with a corner frequency of between 17 is missing parent: 50 hertz and 17 is missing parent: 120 hertz inclusive.

In Example 18, the subject matter of any one or more of Examples 1-17 optionally include wherein to pass the impact data through a corresponding filter to generate filtered impact data includes adjusting the filtered impact data for an estimated attenuation caused by the filter.

In Example 19, the subject matter of Example 18 optionally includes wherein to adjust the filtered impact data for an estimated attenuation caused by the filter includes to multiply the filtered impact data by a multiplication factor determined using a lookup table.

In Example 20, the subject matter of Example 19 optionally includes wherein the lookup table includes a multiplication value based on the type of impact data and a corner frequency of the filter.

In Example 21, the subject matter of any one or more of Examples 1-20 optionally include wherein the one or more features indicative of a noise level include one or more features indicative of a signal-to-noise ratio.

In Example 22, the subject matter of any one or more of Examples 1-21 optionally include wherein the signal receiver circuit and the assessment circuit are included in a wearable mouthguard.

In Example 23, the subject matter of Example 22 optionally includes wherein the wearable mouthguard further comprises a communication circuit configured to transmit a representation of the filtered impact data.

In Example 24, the subject matter of any one or more of Examples 1-23 optionally include milliseconds after the peak of an impact event.

In Example 25, the subject matter of any one or more of Examples 1-24 optionally include wherein for one or more classifications, the corresponding filter generates filtered impact data that matches the impact data.

Example 26 is a monitoring device system, the system comprising: a signal receiver circuit configured to receive kinematic information of a user, the kinematic information including impact data corresponding to a head acceleration event (HAE); and an assessment circuit configured to process the impact data, including to: classify, following determining that the impact data corresponds to an HAE, the HAE based on one or more features indicative of a noise level of the HAE; and based upon the classification, pass the impact data through a corresponding filter to generate filtered impact data.

Example 27 is a method for classifying a head acceleration event (HAE), the method comprising: receiving kinematic information of a user, the kinematic information including impact data corresponding to the HAE; classifying the HAE based on one or more features indicative of a noise level of the HAE; and filtering the impact data through a filter corresponding to the classification to generate filtered impact data.

Example 28 is a monitoring device system including a wearable mouthguard, the system comprising: a signal receiver circuit configured to receive proximity data from a proximity sensor in the wearable mouthguard; and an assessment circuit configured to process proximity data from the wearable mouthguard and determine whether the wearable mouthguard is engaged on the teeth of a user, including to: determine a “on-tooth” value of the proximity sensor; and compare the proximity data to the “on-tooth” value.

In Example 29, the subject matter of Example 28 optionally includes wherein to determine the “on-tooth” value, the assessment circuit is configured to: record a plurality of values of the proximity data; and determine a moving average of the plurality of values.

In Example 30, the subject matter of Example 29 optionally includes wherein to determine a moving average of the plurality of values, the assessment circuit is configured to: generate a histogram including the plurality of values; and select a most populous histogram bin that is above a specified threshold level.

In Example 31, the subject matter of Example 30 optionally includes wherein to determine a moving average of the plurality of values, the assessment circuit is further configured to: generate a plurality of histograms using proximity data from different times; and determine a moving average of the most populous histogram bins.

In Example 32, the subject matter of any one or more of Examples 28-31 optionally include wherein: the signal receiver circuit is configured to receive kinematic information of a user, the kinematic information including impact data corresponding to an impact event; and the assessment circuit is configured to determine whether the impact event is a head acceleration event (HAE), including to: compare proximity data from before and after the impact event to the “on-tooth” value.

In Example 33, the subject matter of Example 32 optionally includes wherein the assessment circuit is configured to: determine if the proximity data from before and after the impact event are within a specified first threshold range of the “on-tooth” value, and when they are: classify the impact even as an HAE.

In Example 34, the subject matter of any one or more of Examples 32-33 optionally include wherein: the assessment circuit is configured to process the impact data when it is determined that the impact event is a HAE, including to: classify the HAE based on one or more features indicative of a noise level of the HAE; and based upon the classification, pass the impact data through a corresponding filter to generate filtered impact data.

In Example 35, the subject matter of any one or more of Examples 29-34 optionally include wherein: the signal receiver circuit is further configured to receive orientation data indicative of an orientation of the wearable mouthguard and kinematic information indicative of a motion of the wearable mouthguard; and to record the plurality of values of the proximity data, the assessment circuit is further configured to process the orientation data and the kinematic information to determine if the wearable mouthguard is upright and generally stationary and when it is, record the plurality of values of the proximity data.

Example 36 is at least one machine-readable medium including instructions that, when executed by processing circuitry, cause the processing circuitry to perform operations to implement of any of Examples 1-35.

Example 37 is an apparatus comprising means to implement of any of Examples 1-35.

Example 38 is a system to implement of any of Examples 1-35.

Example 39 is a method to implement of any of Examples 1-35.

Each of the non-limiting aspects above can stand on its own or can be combined in various permutations or combinations with one or more of the other aspects or other subject matter described in this document.

The above detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the terms “or” and “and/or” are used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4).

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like.

Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A monitoring device system, the system comprising:
a signal receiver circuit configured to receive kinematic information of a user, the kinematic information including impact data corresponding to a head acceleration event (HAE); and
an assessment circuit configured to process the impact data, including to:
classify the HAE based on one or more features indicative of a noise level of the HAE; and
based upon the classification, pass the impact data through a corresponding filter to generate filtered impact data.

2. The system of claim 1, wherein the assessment circuit is further configured to determine a value of the HAE by analyzing the filtered impact data.

3. The system of claim 2, wherein the assessment circuit is further configured to compare the value to a threshold, and, when the value is on a specified side of the threshold, trigger an alert to be generated.

4. The system of claim 2, wherein the value includes at least one of peak linear acceleration, peak angular acceleration, peak angular velocity, peak linear velocity, workload, impact direction, or impact location.

5. The system of claim 2, wherein the value of the HAE is used to determine whether the user can continue participation in a sporting event.

6. The system of claim 1, wherein to classify the HAE based on one or more features indicative of a noise level includes using a machine learning model to classify the HAE.

7. The system of claim 6, wherein the machine learning model has been trained using datasets including classified HAEs.

8. The system of claim 7, wherein the machine learning model includes a support vector machine (SVM), wherein the SVM has been trained to classify HAEs based on their noise level using the one or more features indicative of a noise level of the HAE.

9. The system of claim 8, wherein the one or more features include a plurality of features, wherein one or the plurality of features includes a ratio of the translational, tangential, and centripetal acceleration to a total peak linear acceleration of a center of gravity of a head of the user.

10. The system of claim 6, wherein the machine learning model includes a neural network model, wherein the neural network model has been trained to classify HAEs based on their noise level.

11. The system of claim 6, wherein, before being classified by the machine learning model, it is determined that the impact data corresponds to an HAE.

12. The system of claim 11, wherein the monitoring device system includes a wearable mouthguard, wherein to determine that the impact data corresponds to an HAE, the signal receiver circuit is configured to receive proximity data from a proximity sensor in the wearable mouthguard and the assessment circuit is configured to process proximity data from the wearable mouthguard and determine whether the wearable mouthguard is engaged on the teeth of the user.

13. The system of claim 12, wherein to determine if the wearable mouthguard is engaged on the teeth of the user includes comparing proximity data received before and after the impact data to a threshold range of values.

14. The system of claim 12, wherein to determine that the impact data corresponds to an HAE further includes:
receiving, using the signal receiver circuit, orientation data related to an orientation of the wearable mouthguard; and
determining, using the assessment circuit, whether the orientation data indicates that the wearable mouthguard is in a generally upright orientation.

15. The system of claim 1, wherein to pass the impact data through a corresponding filter comprises passing the impact data through a lowpass filter, wherein a corner frequency of the lowpass filter is higher for HAEs classified as having a lower noise level.

16. The system of claim 15, wherein:
the classifications include a low noise level classification and a high noise level classification;
impact data in the low noise level classification are determined to have a lower noise level than impact data in the high noise level classification;
impact data in the low noise level classification is passed through a lowpass filter with a corner frequency of between 160 hertz and 200 hertz inclusive; and
impact data in the high noise level classification is passed through a lowpass filter with a corner frequency of between 50 hertz and 120 hertz inclusive.

17. The system of claim 1, wherein the signal receiver circuit and the assessment circuit are included in a wearable mouthguard.

18. The system of claim 17, wherein the wearable mouthguard further comprises a communication circuit configured to transmit a representation of the filtered impact data.

19. The system of claim 1, wherein:
Classifying the HAE based on one or more features indicative of a noise level of the HAE is performed following determining that the impact data corresponds to an HAE.

20. A method for classifying a head acceleration event (HAE), the method comprising:
receiving kinematic information of a user, the kinematic information including impact data corresponding to the HAE;
classifying the HAE based on one or more features indicative of a noise level of the HAE; and
filtering the impact data through a filter corresponding to the classification to generate filtered impact data.

21. A monitoring device system including a wearable mouthguard, the system comprising:
a signal receiver circuit configured to receive proximity data from a proximity sensor in the wearable mouthguard; and
an assessment circuit configured to process proximity data from the wearable mouthguard and determine whether the wearable mouthguard is engaged on the teeth of a user, including to:
determine a “on-tooth” value of the proximity sensor; and
compare the proximity data to the “on-tooth” value.

22. The monitoring device system of claim 21, wherein to determine the “on-tooth” value, the assessment circuit is configured to:
record a plurality of values of the proximity data; and
determine a moving average of the plurality of values.
